(19) United States
(12) Patent Application Publication
(10) Pub. No.: US 2014/0349411 A1
(43) Pub. Date: Nov. 27, 2014

(54) METHOD AND SYSTEM FOR DETECTION OF AFLATOXIN

(71) Applicant: Texas A&M University System, College Station, TX (US)

(72) Inventors: Jun ZOU, College Station, TX (US); Youjun DENG, College Station, TX (US); He HU, College Station, TX (US); Alejandro GARCIA-URIBE, St. Louis, MO (US)

(73) Assignee: Texas A&M University System, College Station, TX (US)

(21) Appl. No.: 14/284,774

(22) Filed: May 22, 2014

Related U.S. Application Data
(60) Provisional application No. 61/826,844, filed on May 23, 2013.

(51) Int. Cl. G01N 33/53

(52) U.S. Cl. CPC G01N 33/5308; G01N 2333/38
USPC 436/501; 422/69; 156/60

(57) ABSTRACT

The present invention relates to an aflatoxin-detection device. The aflatoxin-detection device includes a flow path for a test solution and a plurality of nanocompo site strips disposed within the flow path. Each nanocomposite strip of the plurality of nanocomposite strips is arranged in a spaced parallel relationship with a successive nanocomposite strip of the plurality of nanocomposite strips. The plurality of nanocomposite strips exhibit high affinity for aflatoxin. Absorption of aflatoxin induces fluorescence of the plurality of nanocomposite strips. Responsive to a fluorescence intensity of each nanocomposite strip of the plurality of nanocomposite strips, a concentration of aflatoxin in the test solution is determined.

### CROSS-REFERENCE TO RELATED APPLICATIONS

This application claims priority to, and incorporates by reference the entire disclosure of, U.S. Provisional Patent Application No. 61/826,844, filed May 23, 2013.

### BACKGROUND

1. Field of the Invention

The present invention relates generally to sensors for detection of toxins and more particularly, but not by way of limitation, to sensors utilizing a smectite-polymer nanocomposite coating for detection of aflatoxins.

2. History of the Related Art

Aflatoxins, a harmful byproduct of mold, represent a major type of biological toxin responsible for both acutely toxic and carcinogenic effects on humans and animals alike. Contamination of agricultural commodities, human foods, and animal feeds with aflatoxins have resulted in significant concerns for the food industry. Rapid, quantitative, and low-cost detection methods are important for the timely evaluation, monitoring, and mitigation of hazardous effects caused by aflatoxins.

### SUMMARY

The present invention relates generally to sensors for detection of toxins and more particularly, but not by way of limitation, to sensors utilizing a smectite-polymer nanocomposite coating for detection of aflatoxins. In one embodiment, the present invention relates to an aflatoxin-detection device. The aflatoxin-detection device includes a flow path for a test solution and a plurality of nanocomposite strips disposed within the flow path. Each nanocomposite strip of the plurality of nanocomposite strips is arranged in a spaced parallel relationship with a successive nanocomposite strip of the plurality of nanocomposite strips. The plurality of nanocomposite strips exhibit high affinity for aflatoxin. Absorption of aflatoxin induces fluorescence of the plurality of nanocomposite strips. Responsive to a fluorescence intensity of each nanocomposite strip of the plurality of nanocomposite strips, a concentration of aflatoxin in the test solution is determined.

In another embodiment, the present invention relates to a method for detecting aflatoxin. The method includes conducting a test solution through a flow path formed in an aflatoxin-detection device. The flow path includes a plurality of nanocomposite strips formed therein. The method also includes exposing the aflatoxin-detection device to ultraviolet illumination. The ultraviolet illumination induces fluorescence of certain nanocomposite strips of the plurality of nanocomposite strips. Responsive to a fluorescence intensity of the certain nanocomposite strips, a concentration of aflatoxin in the test solution is determined.

In another embodiment, the present invention relates to a method for producing an aflatoxin-detection device. The method includes forming a stencil having a plurality of parallel slots, applying the stencil to a substrate, and applying a plurality of nanocomposite strips to the substrate utilizing the stencil. The method also includes removing the stencil from the substrate, forming a flow layer, and coupling the flow layer to the substrate.

### BRIEF DESCRIPTION OF THE DRAWINGS

For a more complete understanding of the present invention and for further objects and advantages thereof, reference may now be had to the following description taken in conjunction with the accompanying drawings in which:

FIG. 1 is an exploded perspective view of an aflatoxin-detection device according to an exemplary embodiment;

FIG. 2 is a flow diagram of a process for making an aflatoxin-detection device according to an exemplary embodiment;

FIG. 3A is a schematic diagram of an aflatoxin-analysis system according to an exemplary embodiment;

FIG. 3B is a flow diagram of a process for using an aflatoxin-detection device according to an exemplary embodiment;

FIG. 4 is a graph of fluorescence intensity according to an exemplary embodiment.

### DETAILED DESCRIPTION

Various embodiments of the present invention will now be described more fully with reference to the accompanying drawings. The invention may, however, be embodied in many different forms and should not be construed as limited to the embodiments set forth herein.

Aflatoxin detection is currently performed using high-performance liquid chromatography (“HPLC”) followed by fluorometric or mass spectroscopic analysis. This is a very time consuming and costly procedure and, as a result, has been primarily limited to laboratory use. A number of rapid-detection methods based on immunoassays have also been developed. These rapid-detection methods utilize antibodies to selectively capture aflatoxins in a test solution. These rapid-detection methods have limitations. First, they are susceptible to denaturation and degradation and, as a result, require very strict testing conditions for their effective functioning. Second, the production of antibodies requires live animal and can be a complex and expensive process.

Several bentonites (smectite-rich clays) have been used as adsorbent additives to detoxify aflatoxin-contaminated animal feeds. Recent studies have demonstrated that divalent cations and transition cations in the interlayers of smectite can induce the substantial bonding of the aflatoxin to the smectite. Unlike antibodies, the smectite-aflatoxin binding is hardly affected by various adsorption conditions such as, for example, temperature or pH value. In addition, a high adsorption capacity such as, for example, 11>-20% of the self weight of the smectite can also be obtained due to the large surface area (about 800 m2/g) of the smectite interlayers. Because of its high absorption selectivity and capacity for aflatoxin, smectite could be developed into a new molecular recognition agent for the aflatoxin detection, serving as an inexpensive inorganic substitute for the delicate and costly antibodies.

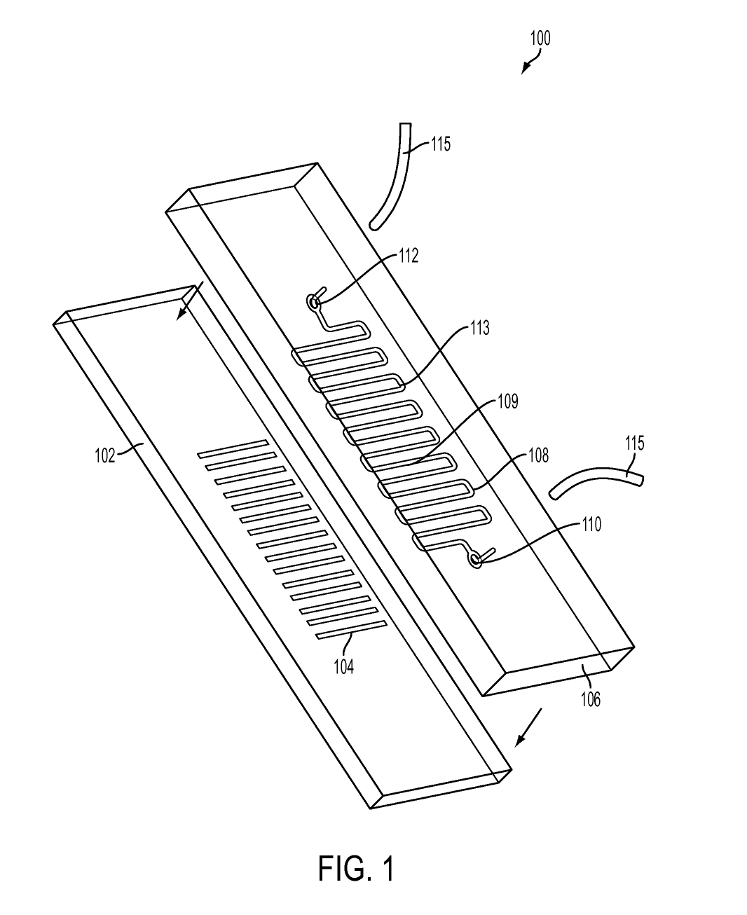

FIG. 1 is an exploded perspective view of an aflatoxin-detection device 100. The aflatoxin-detection device 100 includes a substrate layer 102 that is coupled to a flow layer 106. In a typical embodiment, the substrate layer 102 is, for example, a glass microscope slide, however, other appropriate materials may be utilized. A plurality of nanocomposite strips 104 are disposed on a surface of the substrate layer 102 at regular-spaced intervals. In a typical embodiment, the nanocomposite strips 104 are constructed of a clay mineral such as, for example, Smectite-polyacrylamide. The flow layer 106 includes a flow path 108 formed in a surface facing the substrate layer 102. In a typical embodiment, the flow path 108 in formed through a process such as, for example, photo-lithography, and has a depth of approximately 10 μm. The flow path 108 includes a plurality of parallel sections 109. The plurality of parallel sections 109 are fluidly coupled by bends 113. An inlet port 110 and an outlet port 112 are fluidly coupled to respective ends of the flow path 108. Tubing 115 such as, for example, polyamide tubing is fluidly coupled to the inlet port 110 and the outlet port 112. When the flow layer 106 is assembled to the substrate layer 102, the nanocomposite strips 104 are enclosed within the flow path 108.

Still referring to FIG. 1, in a typical embodiment, the nanocomposite strips 104 are synthesized on flat silicon substrates via a layer-by-layer assembling process utilizing 1 g/L polyacrylamide aqueous solution and 1 g/L Smectite dispersion. A group of pre-cleaned silicon substrates are immersed into the polyacrylamide aqueous solution for, for example, seven minutes and then rinsed in deionized water. Next the silicon substrates are immersed in the Smectite dispersion for, for example, five minutes and then rinsed in deionized water. This cycle is repeated until the silicon substrate is fully covered with a nanocomposite film. By way of example, the optimal polyacrylamide concentration has been shown to be approximately 0.005%.

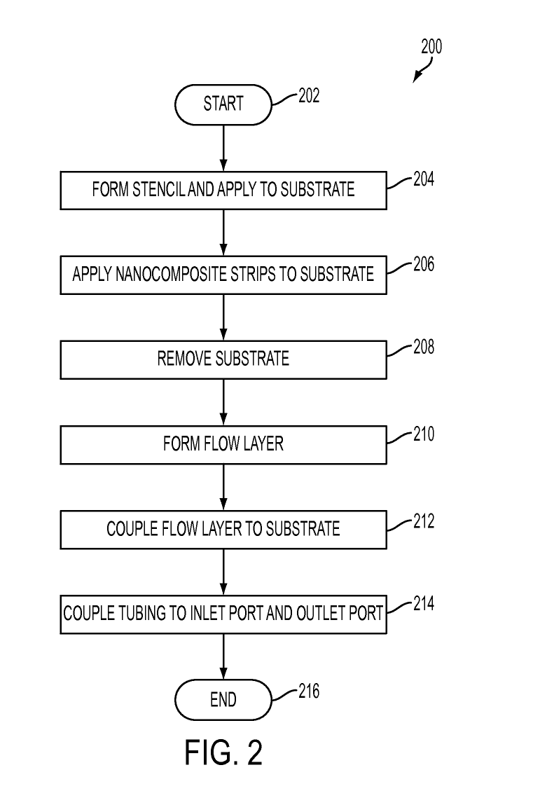

FIG. 2 is a flow diagram of a process 200 for making the aflatoxin-detection device 100. The process 200 starts at step 202. At step 204, a stencil is formed from a transparency sheet. The stencil includes a plurality of regular-spaced generally-parallel slots. The stencil is applied to the substrate layer 102 and secured thereto via, for example, a binder clip. At step 206, the nanocomposite strips 104 are applied to the substrate layer 102 utilizing the stencil. The nanocomposite strips 104 are applied in the regular-spaced generally parallel slots of the stencil. At step 208, the stencil is removed from the substrate layer 102 leaving the nanocomposite strips 104 properly located on the substrate layer 102. At step 210 the flow layer 106 is formed. A flow-path pattern is formed on, for example, a silicon wafer. The flow path 108 is formed then formed in the flow layer 106 via, for example, photolithography. At step 212, the flow layer 106 is coupled to the substrate layer 106 such that the nanocomposite strips 104 are enclosed in the parallel sections 109 of the flow path 108. At step 214, the tubing 214, such as, for example, polyamide tubing, is fluidly coupled to the inlet port 110 and the outlet port 112. The process 200 ends at step 216.

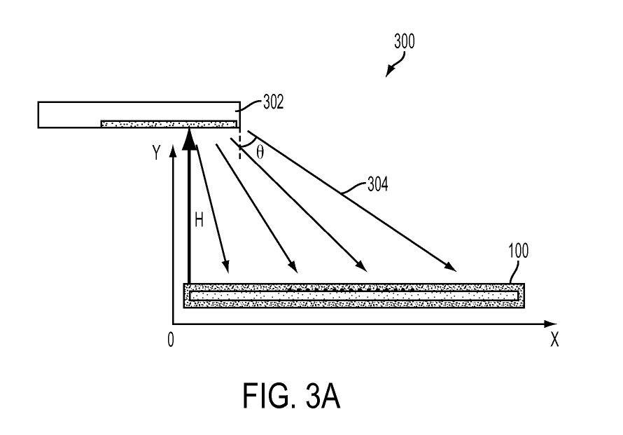

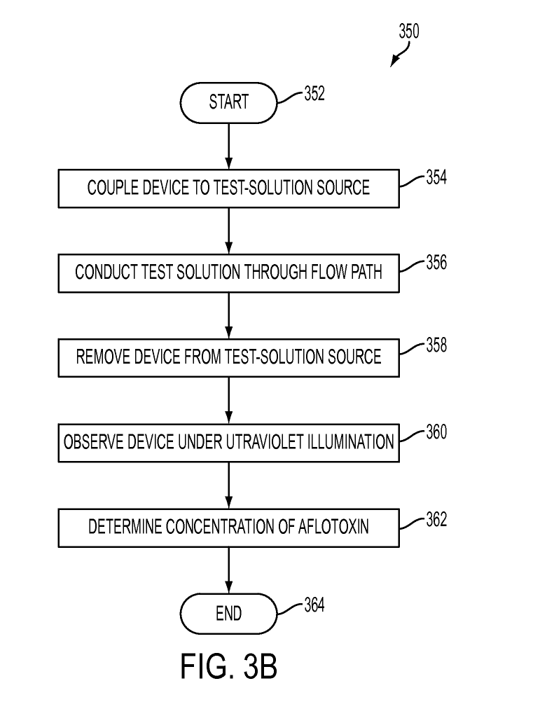

FIG. 3A is a schematic diagram of an aflatoxin-analysis system 300. The aflatoxin-analysis system 300 includes an ultraviolet lamp 302. The aflatoxin-detection device 100 is positioned under the ultraviolet lamp 302 such that ultraviolet light 304 emitted from the ultraviolet lamp 302 is incident upon the aflatoxin-detection device 100 at an angle (θ).

Still referring to FIG. 3A, fluorescence intensity from aflatoxin adsorbed to the nanocomposite strips 104 is proportional to the concentration of aflatoxins in the test solution. Thus, under normal ultraviolet illumination (θ=0 degrees), uniform fluorescence emission will be observed from the nanocomposite strips 104. As shown in FIG. 3A, under oblique illumination (θ>0 degrees), as a distance between a nanocomposite strip 104 increases, a fluorescence intensity emitted from the nanocomposite strip 104 decreases. In this scenario, the average excitation intensity on a nanocomposite strip (x) is expressed in formula 1 below:

$$I ( x ) = \frac{{I}_{0}}{{h}^{2} + {x}^{2}} \qquad Formula 1$$

Where I(x) is the is the excitation intensity of the nanocomposite strip (x), I₀ is the intensity of the ultraviolet lamp 302, h is the vertical distance between the aflatoxin-detection device 100 and the ultraviolet lamp 302, and x is the horizontal distance between the ultraviolet lamp 302 and the nanocomposite strip (x).

The fluorescence intensity of the nanocomposite strip (x) is expressed in formula 2 below:

$${I}_{fi} ( x ) ∝ {C}_{i} \frac{{I}_{0}}{{h}^{2} + {x}^{2}} \qquad Formula 2$$

Where I_{fi} is a fluorescence intensity of the nanocomposite strip (x) and Cᵢ is a concentration of aflatoxin in the test solution. This correlation makes it possible to achieve a quantitative determination of aflatoxin concentration in the test solution by counting a number of fluorescing nanocomposite strips.

FIG. 3B is a flow diagram of a process 350 for using the aflatoxin-detection device 100. The process starts at step 352. At step 354, the aflatoxin-detection device is fluidly coupled to a test-solution source. In a typical embodiment, the testing solution is conducted to the aflatoxin-detection device via the tubing 115 such as, for example, polyamide tubing. The tubing is fluidly coupled to the inlet port 110 and the outlet port 112 of the aflatoxin-detection device. At step 356, the testing solution is conducted through the flow path 108 of the aflatoxin-detection device 100. The testing solution may be conducted through the flow path 108 for a period of time of approximately 2 minutes up to approximately 20 minutes or more.

The nanocomposite strips 104 absorb molecules of aflatoxin that are present in the test solution. Absorption of aflatoxin molecules results in a highly concentrated accumulation of aflatoxin molecules in the nanocomposite strips 104. At step 360, the aflatoxin-detection device 100 is observed under ultraviolet illumination and a fluorescent intensity of the nanocomposite strips 104 is observed. At step 362, a concentration of aflatoxin present in the test solution is determined based upon the fluorescent intensity of the nanocomposite strips 104. The process 350 ends at step 364.

In a typical embodiment, when the aflatoxin-detection device 100 is illuminated under oblique ultraviolet illumination, a fluorescence intensity of the nanocomposite strips 104 decreases as a distance from the ultraviolet lamp 302 increases. Oblique ultraviolet illumination creates a non-uniform illumination field with a large gradient along a length of the aflatoxin-detection device 100. The aflatoxin-detection device 100 exhibits high sensitivity and linearity. The nanocomposite strips 104 exhibit a high affinity for aflatoxin molecules thus giving the aflatoxin-detection device 100 a high degree of sensitivity. Further, because the fluorescence intensity of aflatoxin is proportional to the concentration of aflatoxin, the aflatoxin-detection device 100 also provides a high-degree of linearity for aflatoxin detection.

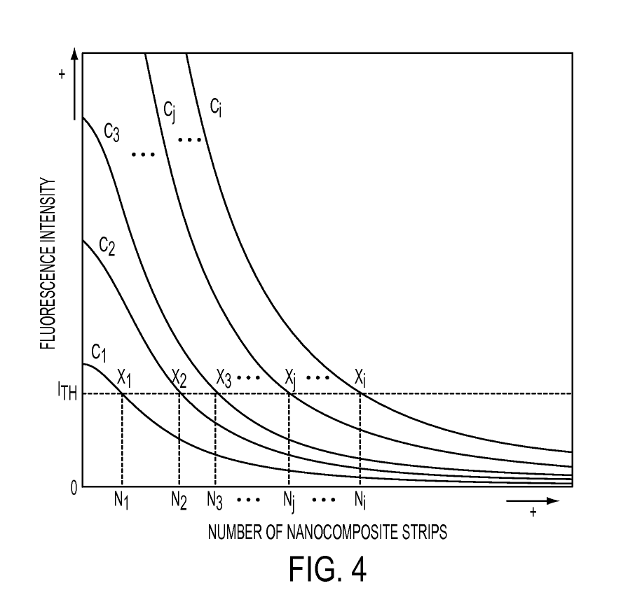

FIG. 4 is a graph of fluorescence intensity of the nanocomposite strips 104 according to an exemplary embodiment. A threshold fluorescence intensity Ith represents a weakest fluorescence that can be visually distinguished. An intercept point between I f i (x) curves and the Ith line determines a number of nanocomposite strips 104 (Nj), which can be effectively observed visually. Higher aflatoxin concentration in the test solution leads to more adsorption on the nanocomposite strips 104 and a larger number of observable nanocomposite strips 104. This correlation makes it possible to achieve a quantitative estimation of aflatoxin concentration in the test solution by just counting the number of “fluorescing” nanocomposite strips 104 without involving sophisticated spectrofluorometers.

High absorption capacity of the nanocomposite strips 104 allows the aflatoxin-detection device 100 to detect very low levels of aflatoxin such as, for example, in the range of approximately 10 parts per billion. Furthermore, the nanocomposite strips 104 are unaffected by the presence of other organic or inorganic compounds. The nanocomposite strips 104 also exhibit structural and chemical stability, thereby allowing the aflatoxin-detection device 100 to have a long shelf life. Finally, the aflatoxin detection device 100 allows detection of aflatoxin in a period of time of, for example, approximately 10 minutes or less.

Although various embodiments of the method and system of the present invention have been illustrated in the accompanying Drawings and described in the foregoing Specification, it will be understood that the invention is not limited to the embodiments disclosed, but is capable of numerous rearrangements, modifications, and substitutions without departing from the spirit and scope of the invention as set forth herein. It is intended that the Specification and examples be considered as illustrative only.

What is claimed is:

1. An aflatoxin-detection device comprising:
a flow path for a test solution;
a plurality of nanocomposite strips disposed within the flow path, each nanocomposite strip of the plurality of nanocomposite strips being arranged in a spaced parallel relationship with a successive nanocomposite strip of the plurality of nanocomposite strips;
wherein absorption of aflatoxin by the plurality of nanocomposite strips induces fluorescence of the plurality of nanocomposite strips; and
wherein, responsive to a fluorescence intensity of each nanocomposite strip of the plurality of nanocomposite strips, a concentration of aflatoxin in the test solution is determined.

2. The aflatoxin-detection device according to claim 1, wherein the flow path is a serpentine flow path comprising a plurality of parallel segments.

3. The aflatoxin-detection device according to claim 2, wherein each nanocomposite strip of the plurality of nanocomposite strips is arranged in a parallel segment of the plurality of parallel segments.

4. The aflatoxin-detection device according to claim 1, wherein the plurality of nanocomposite strips fluoresce responsive to absorption of aflatoxin.

5. The aflatoxin-detection device according to claim 1, wherein the plurality of nanocomposite strips comprise Smectite-polyacrylamide.

6. The aflatoxin-detection device according to claim 1, comprising:
a substrate layer having the plurality of nanocomposite strips formed thereon; and
a flow layer having the flow path formed therein.

7. A method for detecting aflatoxin, the method comprising:
conducting a test solution through a flow path formed in an aflatoxin-detection device, the flow path comprising a plurality of nanocomposite strips formed therein;
exposing the aflatoxin-detection device to ultraviolet illumination, the ultraviolet illumination inducing fluorescence of certain nanocomposite strips of the plurality of nanocomposite strips; and
responsive to a fluorescence intensity of the certain nanocomposite strips, determining a concentration of aflatoxin in the test solution.

8. The method of claim 7, wherein the exposing comprises exposing the aflatoxin-detection device to oblique ultraviolet illumination.

9. The method of claim 7, wherein the plurality of nanocomposite strips comprise Smectite-polyacrylamide.

10. The method of claim 7, wherein a concentration of aflatoxin determines a degree of fluorescence of each nanocomposite strip of the plurality of nanocomposite strips.

11. The method of claim 7, wherein the determining comprises counting a number of fluorescing nanocomposite strips.

12. The method of claim 11, wherein a higher concentration of aflatoxin results in a greater number of fluorescing nanocomposite strips.

13. The method of claim 7, wherein the test solution is conducted through the flow path for a period in a range of approximately 2 minutes to approximately 20 minutes.

14. The method of claim 7, wherein the nanocomposite strips induce bonding of aflatoxin.

15. The method of claim 14, wherein bonding of the aflatoxin to the nanocomposite strips is unaffected by temperature and pH.

16. The method of claim 7, wherein the aflatoxin-detection device detects aflatoxin in a range of approximately 10 parts per billion.

17. A method for producing an aflatoxin-detection device, the method comprising:
forming a stencil having a plurality of parallel slots;
applying the stencil to a substrate;
applying a plurality of nanocomposite strips to the substrate utilizing the stencil;
removing the stencil from the substrate;
forming a flow layer; and
coupling the flow layer to the substrate.

18. The method of claim 17, wherein the plurality of nanocomposite strips comprise Smectite-polyacrylamide.

19. The method of claim 17, wherein the flow layer comprises a serpentine flow path comprising a plurality of parallel segments.

20. The method of claim 19, wherein each nanocomposite strip of the plurality of nanocomposite strips is arranged in a parallel segment of the plurality of parallel segments.
